(19) United States
(12) Patent Application Publication
(10) Pub. No.: US 2017/0049497 A1
(43) Pub. Date: Feb. 23, 2017

(54) ARRAY ORIENTATION TRACKER

(71) Applicant: Medtronic Ablation Frontiers LLC, Minneapolis, MN (US)

(72) Inventor: Monica M. RADECKE, Stillwater, MN (US)

(21) Appl. No.: 14/832,461

(22) Filed: Aug. 21, 2015

(51) Int. Cl. A61B 18/14; A61B 19/00; A61B 18/12

(52) U.S. Cl. CPC A61B 18/14; A61B 18/1206; A61B 19/46; A61B 19/50; A61B 2018/00577

(57) ABSTRACT

A device, system, and method for predicting or determining lesion efficacy and method for displaying lesion efficacy data to a user. The device may include a treatment assembly including a plurality of electrodes and a location tracking element, such as a gyroscope or accelerometer. The location tracking element may determine an angular velocity and/or linear acceleration of the treatment assembly as the treatment assembly is rotated and moved, and the velocity data may be used to determine an angle of rotation, which may be used to determine the location of each of the plurality of electrodes relative to an area of tissue. As the tissue is ablated with the plurality of electrodes, a lesion prediction plot may be displayed to the user. The lesion prediction plot may include one or more graphical representations indicating a total number of effective contact seconds between each electrode and the tissue.

### CROSS-REFERENCE TO RELATED APPLICATION

n/a

### STATEMENT REGARDING FEDERALLY SPONSORED RESEARCH OR DEVELOPMENT

n/a

### TECHNICAL FIELD

The present invention relates to a device, system, and method for predicting or determining device orientation. The present invention further relates to a device, system, and method for displaying lesion data to a user when paired with orientation information. Further, the present invention broadly relates to any other type of data that can be coupled with orientation such as contact sensing or energy delivery.

### BACKGROUND

A cardiac arrhythmia is a condition in which the heart's normal rhythm is disrupted. Certain types of cardiac arrhythmias, including ventricular tachycardia and atrial fibrillation, may be treated by ablation (for example, radiofrequency (RF) ablation, cryoablation, ultrasound ablation, laser ablation, microwave ablation, electroporation, and the like), either endocardially or epicardially.

Procedures such as pulmonary vein isolation (PVI) and pulmonary vein antrum isolation (PVAI) are commonly used to treat atrial fibrillation. These procedures generally involve the use of an ablation device, such as a catheter, with which the treatment delivery segment is positioned at the ostium of a pulmonary vein (PV) such that any blood flow exiting the PV into the left atrium (LA) may be completely blocked, depending on the device. Once in position, the ablation device may be activated for a sufficient duration to create a desired lesion within myocardial tissue at the PV-LA junction, such as a PV ostium or PV antrum. For example, the device may include a cryoballoon, single tip electrode, or multi-electrode array that is used as the treatment element of the device to create a circumferential lesion about the ostium and/or antrum of the PV to disrupt aberrant electrical signals exiting the PV.

The success of this procedure depends largely on the quality of the lesion(s) created during the procedure to achieve pulmonary vein isolation. There are several ways in which lesion formation may be assessed, either during or after an ablation procedure. Such methods include pacing maneuvers, navigation, imaging techniques, temperature measurement, temperature-time assessment, and cell-death models. However, each of these methods has its drawbacks. For example, pacing maneuvers performed between treatments of each vein may be useful for electrical evaluation of pulmonary vein isolation, but provide no image of the lesion. Further, navigation systems can “map” lesion sets, but not all catheters are compatible and it adds procedure time and cost. Additionally, standard imaging techniques may involve interrupting the ablation procedure to image the target tissue to determine if further ablation is required for sufficient lesion creation. This post-procedural medical imaging does not provide real-time lesion assessment and/or prevention of injury to non-target tissue. Further, the cell-death models may not be very robust, may over- or under-estimate the effects an ablation procedure has on the target tissue, and different models may have to be used for different tissue.

It has been determined that real-time lesion formation assessment, anatomical navigation, lesion tagging, and a full procedure summary are some of the most important unmet needs to support safety and efficacy of tissue ablation. None of the methods described above provides feedback that can present dynamic rotational information that can be used to assess lesion formation and other ablation data in real time or that can be used to provide an accurate ablation summary.

Therefore, it is desirable to provide a tissue treatment system and device that allows for the real-time lesion formation assessment, lesion efficacy evaluation, procedure summary, and other ablation information coupled with device orientation.

### SUMMARY

The present invention advantageously provides a device, system, and method for predicting or determining lesion efficacy and method for displaying lesion efficacy data to a user. An ablation device may generally include an elongate body having a proximal portion and a distal portion, a treatment element at the distal portion, and a gyroscope configured to determine an angular velocity of the treatment element. The treatment element may include a carrier arm and a plurality of electrodes, each of the plurality of electrodes being coupled to the carrier arm, and the carrier arm may be configurable into an expanded configuration. Alternatively, the treatment element may include at least two carrier arms, the plurality of electrodes being coupled to each of the at least two carrier arms. The ablation device may further include a shaft that is slidably disposed within the elongate body, the shaft including a distal portion and a proximal portion. The distal portion of the shaft may include a distal tip, the gyroscope being located within the distal tip. The device may further include an accelerometer, the gyroscope and the accelerometer being part of a microprocessing unit. The device may further include a handle at the proximal portion of the ablation device, wherein the microprocessing unit may be located at one of the distal tip of the shaft, a location proximate the at least one electrode, and the handle.

A system for evaluating lesion formation in an area of tissue may generally include an ablation device including: a treatment assembly having at least one electrode; and a location tracking element configured to determine an angular velocity and linear acceleration of at least one of the treatment element and the ablation device. The system may further include a console including: a power generator in electrical communication with the treatment assembly; at least one processor in communication with the power generator and the location tracking element, the processor programmed to: receive ablation data from the power generator; receive at least one of angular velocity data and linear acceleration data from the location tracking element; calculate an angle of rotation of the treatment assembly; and determine a location and movement of the at least one of the treatment assembly and the ablation device. The treatment assembly may further have a carrier arm and the at least one electrode may be a plurality of electrodes, the plurality of electrodes being coupled to the carrier arm. The location tracking element may be coupled to the carrier arm. Alternatively, the ablation device may further include an elongate body having a longitudinal axis, a proximal portion, and a distal portion; a shaft that is slidably movable within and coaxial with the elongate body, the shaft having a proximal portion and a distal portion having a distal tip; and a handle coupled to the proximal portion of the elongate body, and the location tracking element may be located within the distal tip of the shaft or may be coupled to the handle. The location tracking element may be a gyroscope or a gyroscope and an accelerometer included in the same microprocessing unit. The at least one electrode may be a plurality of electrodes and the processor may be further programmed to determine a number of effective contact seconds for each of the plurality of electrodes and to generate a graphical representation of the treatment assembly and the number of effective contact seconds for each of the plurality of electrodes. The processor may be further programmed to receive ablation data from the at least one electrode and to generate a graphical representation of the treatment assembly and ablation data for the at least one electrode.

A method for evaluating energy delivery to an area of tissue may generally include: positioning a treatment assembly of a medical device at an area of tissue, the treatment assembly including a plurality of electrodes, the medical device including a location tracking element configured to determine an angular velocity of the treatment assembly; determining an orientation of the treatment assembly in relation to the area of tissue, the determination being based at least in part on the angular velocity of the treatment assembly; delivering ablation energy from the plurality of electrodes to the area of tissue for a first ablation cycle; determining a number of effective contact seconds between each of the plurality of electrodes and the area of tissue from the first ablation cycle; and generating a lesion prediction display based on the number of effective contact seconds of each of the plurality of electrodes and the area of tissue from the first ablation cycle. For example, the lesion prediction display may include a plurality of isolines, each isoline corresponding to a range of effective contact seconds. The method may further include: repositioning the treatment assembly such that at least one of the plurality of electrodes is in contact with a portion of the area of tissue for which a number of effective contact seconds from the first ablation cycle is below a threshold number; delivering ablation energy from the plurality of electrodes to the area of tissue for a second ablation cycle; determining a number of effective contact seconds between each of the plurality of electrodes and the area of tissue from the second ablation cycle; and updating the lesion prediction display based on a total number of effective contact seconds of each of the plurality of electrodes and the area of tissue from the first ablation cycle and the second ablation cycle. The method may further include correlating each of the isolines to a lesion efficacy score. Further, each second of ablation time for which one of the plurality of electrodes is operating between 50° C. and 65° C. and at at least 3 Watts may be counted as an effective contact second. The location tracking element may include a gyroscope and, optionally, may further include an accelerometer configured to determine a linear acceleration of the treatment assembly.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1A shows an exemplary treatment system configured to evaluate energy delivery and/or lesion creation efficacy, the device having a location tracking element at the distal tip;

FIG. 1B shows an exemplary treatment system configured to evaluate energy delivery and/or lesion creation efficacy, the device having a location tracking element on the handle;

FIGS. 2A and 2B show stylized posterior views of an exemplary treatment catheter in a first rotational position and a second rotational position;

FIG. 3 shows exemplary raw gyroscope data;

FIG. 4 shows an exemplary treatment array image; and

FIG. 5 shows an exemplary graphical representation of a Lesion Prediction Plot (LPP) for an electrode array.

### DETAILED DESCRIPTION

The present invention provides a device, system, and method for determining orientation, optionally in conjunction with location information, of a treatment element of a medical device. This information may be coupled with therapy data and used to predict or determine lesion efficacy and may be processed for displaying any ablation data or lesion efficacy data to a user.

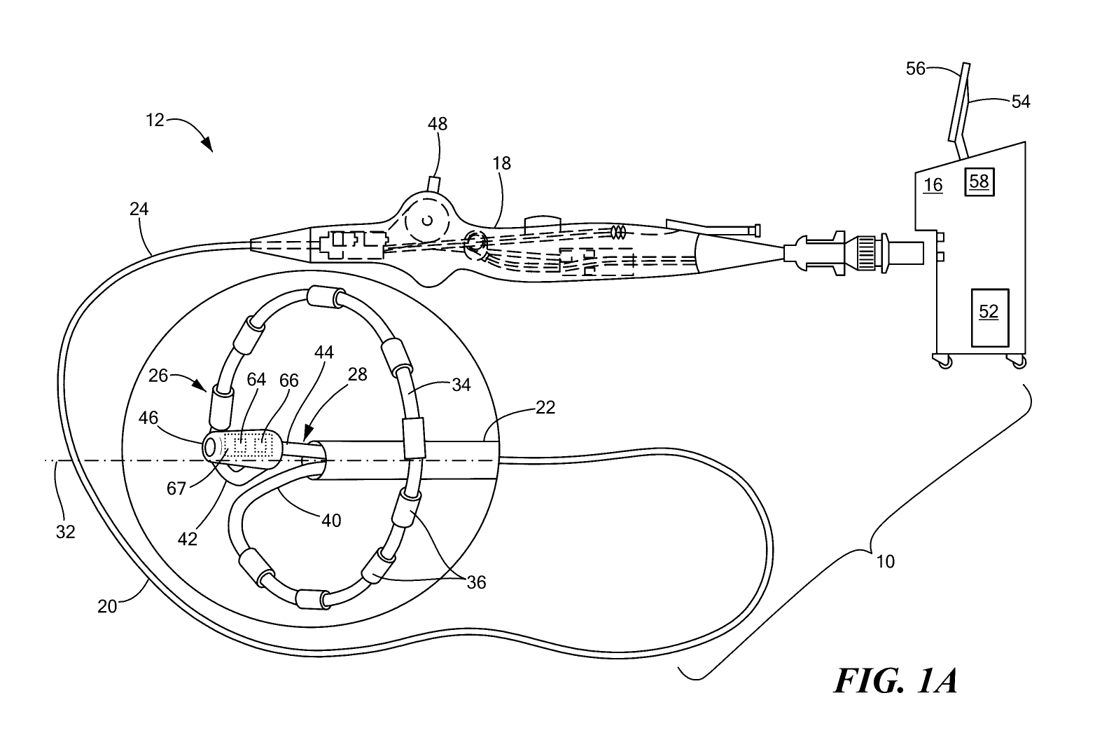

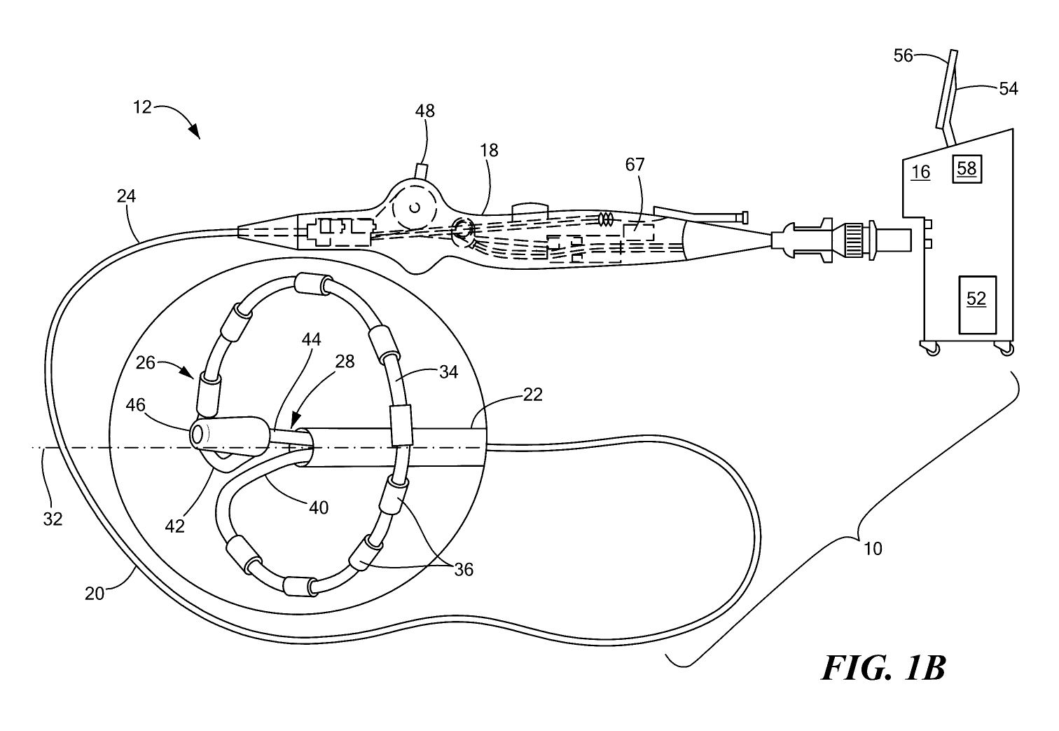

Referring now to FIGS. 1A and 1B, an exemplary treatment system is shown. The system 10 may generally include a treatment device, such as a treatment catheter 12, having one or more location tracking elements, for thermally treating an area of tissue and a console 16 that houses various system 10 controls. The system 10 may be adapted for radiofrequency (RF) ablation and/or phased RF ablation, cryoablation, ultrasound ablation, laser ablation, microwave ablation, hot balloon ablation, or other ablation methods or combinations thereof.

The treatment catheter 12 may generally include a handle 18, an elongate body 20 having a distal portion 22 and a proximal portion 24, one or more treatment elements such as an electrode array 26, and a shaft 28. Further, the treatment catheter 12 may have a longitudinal axis 32. As shown in FIGS. 1A and 1B, the treatment catheter 12 may include an electrode array 26 including a carrier arm 34 bearing a plurality of electrodes 36. The carrier arm 34 may include a first end portion 40 and a second end portion 42, with the first end portion 40 coupled to the distal portion 22 of the elongate body 20 and the second end portion 42 coupled to a distal portion 44 of the shaft 28. Alternatively, the treatment element may include a single electrode, an expandable element with or without electrodes, or any other suitable means for thermally and/or electrically affecting tissue. The distal portion 44 of the shaft 28 may include a distal tip 46 or “nose” that defines a distalmost portion of the treatment catheter 12.

The shaft 28 may lie along the longitudinal axis 32 and be longitudinally movable within the elongate body 20. Further, the carrier arm 34 may be flexible, such that longitudinal movement of the shaft 28 may affect the shape of the carrier arm 34. For example, advancing the shaft 28 distally within the elongate body 20 may cause the electrode array 26 to have a linear or at least substantially linear configuration (not shown), whereas retracting the shaft 28 proximally within the elongate body 20 may cause the electrode array 26 to have a circular or semi-circular configuration (as shown in FIGS. 1A and 1B). The proximal portion of the shaft 28 may be in mechanical communication with one or more steering mechanisms 48 in the handle 18 of the treatment catheter 12, such that the shaft 28 may be longitudinally extended or refracted using one or more steering mechanisms 48, such as knobs, levers, wheels, pull cords, and the like.

In addition to the guide wire lumen 28, the treatment catheter 12 may include one or more lumens. For example, the treatment catheter 12 may include one or more lumens for electrical wiring, steering elements, or the like. Although not shown in the figures, the system 10 may be used for cryotreatment procedures in which tissue is thermally affected by the circulation of a coolant within the treatment element. For example, the one or more treatment elements may include a cryoballoon with or without a plurality of electrodes for ablating tissue. In this case, the treatment catheter 12 may also include, for example, a fluid injection lumen in fluid communication with a coolant supply reservoir and a coolant recovery lumen in fluid communication with a coolant recovery reservoir. Further, the coolant recovery lumen may be in communication with a vacuum to facilitate removal of fluid from the cryoballoon (for example, expanded coolant). It will be understood, therefore, that reference herein to delivering energy to tissue also includes removing heat from tissue through a cryotreatment procedure.

The console 16 may be in electrical and/or fluid communication with the treatment catheter 12 and may include one or more fluid (for example, cryotreatment coolant) reservoirs and coolant recovery reservoirs (not shown), energy generators 52, and computers 54 with displays 56, and may further include various other displays, screens, user input controls, keyboards, buttons, valves, conduits, connectors, power sources, processors, and computers for adjusting and monitoring system 10 parameters. As used herein, the term “computer” may refer to any programmable data-processing unit, including a smart phone, dedicated internal circuitry, user control device, or the like. As a non-limiting example, the system may include a GENius® Generator (Medtronic, Inc.) as an energy generator 52, but the GENius® Generator may also record data from the device, and therefore also be referred to as a “computer.” The computer(s) 54, power generator 52, and/or console 16 may include one or more processors 58 that are in electrical communication with the one or more electrodes 36, the one or more location tracking elements, and one or more fluid valves (for example, if the system 10 is configured for cryotreatment), and may be programmed or programmable to receive data from the treatment catheter 12 (for example, from the one or more location tracking elements), execute an algorithm to determine the orientation and/or location of one or more electrodes 36 of the electrode array 26 (or the cryoballoon), store historical orientation and/or location information, summarize and/or average real-time and/or historical data, and/or to assign a Lesion Efficacy Score (LES) based on this data (as discussed in more detail below). The one or more processors 58 may also be programmed or programmable to generate one or more displays or alerts to notify the user of various system criteria or determinations.

The one or more location tracking elements may include one or more gyroscopes 64 and/or one or more accelerometers 66. Optionally, one or more gyroscopes 64 and one or more accelerometers 66 may be included in one microprocessing unit (MPU) 67, in which case the microprocessing unit may be generally referred to as the location tracking element. For example, the treatment catheter 12 may include a micro electromechanical system (MEMS)/piezoelectric gyroscope 64, which may be located proximate the electrode array 26, such as within the distal tip 46 of the shaft 28. However, the one or more gyroscopes 64 and/or the one or more accelerometers 66 may be located in other places, such as on the carrier arm 34 or on the handle 18 of the treatment catheter 12, and may be small enough that it does not affect device function. Further, if it is desired to only know rotational information of the device, a gyroscope alone may be used; however, processing of the gyroscope data is often coupled with an accelerometer to increase the accuracy of the sensor. That is, the accuracy of the data may be increased with processing algorithms that use information from both the gyroscope 64 and accelerometer 66. For example, if the location tracking element is located proximate the treatment element (for example, as shown in FIG. 1A), data from the gyroscope 64 may be used to track orientation and rotation of the treatment element and the accelerometer 66 data may be used to increase the accuracy of the gyroscope algorithms as well as provide location information of the treatment element. Contrarily, if the location tracking element is located at the handle 18 of the device (for example, as shown in FIG. 1B), data from the gyroscope 64 may be used to track orientation and rotation of the treatment element and the accelerometer data may be used to increase the accuracy of the gyroscope algorithms, but may not be used for location information of the treatment element because of the location tracking element's location farther from the treatment element. Thus, the one or more location tracking elements may be added to almost any make and model of existing device as an after-market add-on component. Additionally, the one or more location tracking elements may add navigation compatibility to a navigation-incompatible device, if the one or more location tracking elements are positioned near the treatment element. For example, by taking data streaming from the accelerometer, when the sensor is located near the treatment element of the device, linear acceleration data can be collected, processed into movement and location data, and used to map the treatment area. If one or more location tracking elements are not positioned along the catheter's longitudinal axis 32, or if they are at a location that is a distance from the electrode array 26 (such as on the handle 18), shear effects, torque effects, and other considerations may have to be incorporated into the calculations disclosed herein.

The gyroscope 64 may track angular velocity around an axis according to the following formula:

$$ω = \frac{d θ}{d t} , \qquad ( 1 )$$

where ω is the angular velocity, θ is the degrees, and t is time. The gyroscope 64 may be a one-axis gyroscope to a three-axis gyroscope. A one-axis gyroscope may provide information about electrode orientation by tracking angular velocity around the axis on which the treatment element is rotated, which may be the same as the longitudinal axis 32 (as shown in FIGS. 1A and 1B). In this manner the angle of each electrode 36 may be tracked relative to its last angle as the device is rotated during a procedure. Further, data from the gyroscope 64 may be used by the one or more processors 58 to rotate a graphical representation of the treatment element, to which a rotational transformation matrix may be applied to visualize the treatment element's new orientation.

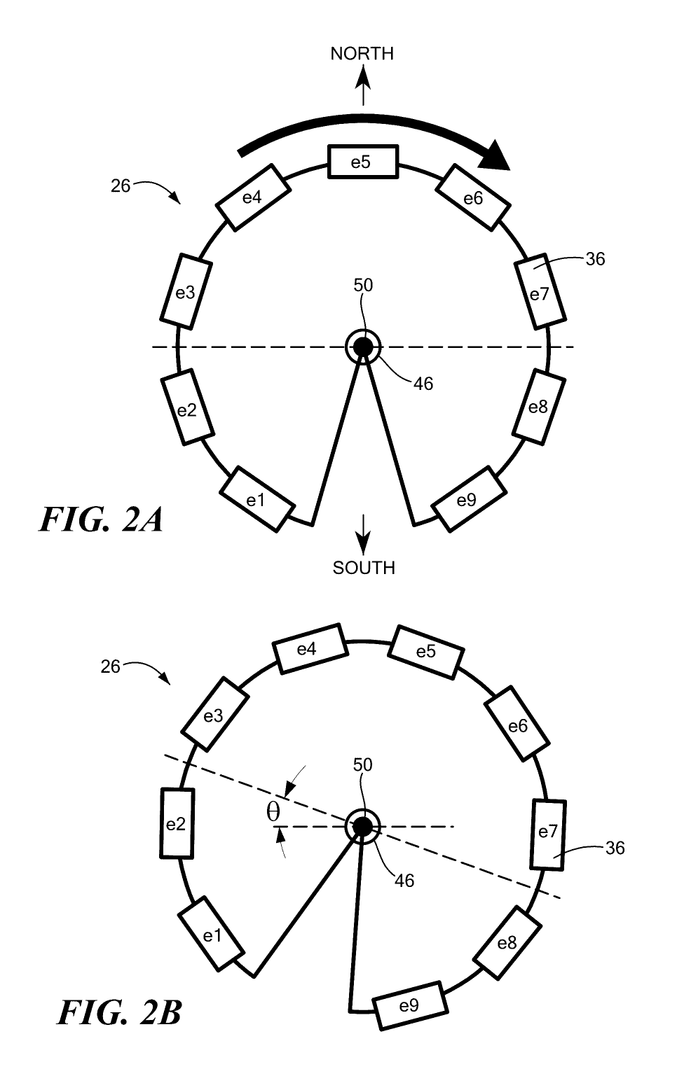

Referring now to FIGS. 2A and 2B, stylized posterior views of an exemplary treatment catheter are shown, with nine electrodes labeled e1-e9. As shown in FIG. 2A, the treatment catheter 12 may include a gyroscope 64 within the distal tip 46 of the shaft 28 (however, any location tracking element may be used). When a user rotates the electrode array 26 by an angle θ from a first orientation to a second orientation, the gyroscope 50 becomes out of plane, which may indicate to the one or more processors 58 that the electrodes 36 are each in a different location. The new two-dimensional coordinates of the electrodes 36 may be calculated based on the angle of rotation θ and the angular velocity ω recorded by the gyroscope 64. Alternatively, three-dimensional accelerometer data may also be used, which may offer orientation information as well as three-dimensional (3D) positional or movement information.

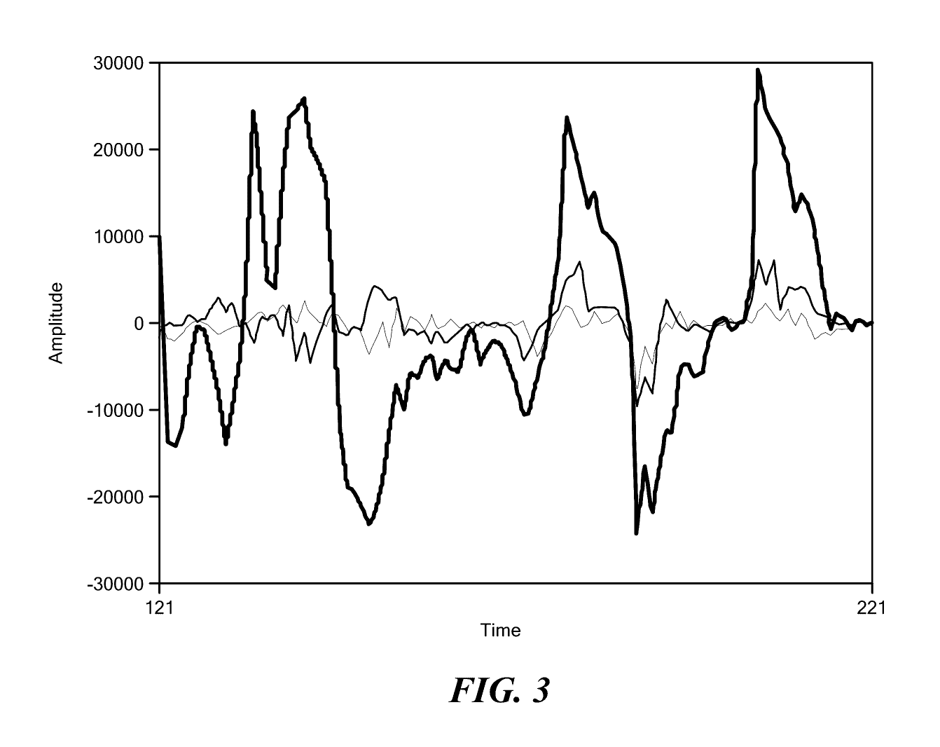

FIG. 3 shows exemplary raw data from a three-axis gyroscope 64. As discussed above, the gyroscope 64 may record angular velocity ω, and this value may be used by the one or more processors 58 to calculate the angle θ of rotation, often in conjunction with a scaling factor intrinsic to the one or more location tracking elements. For example, the following formulae may be used:

$$\begin{matrix}ω = \dot{θ} = \frac{d θ}{d t} * \frac{1}{scaling factor} & ( 2 ) \\ θ ( t ) = {\int }_{0}^{t} \dot{θ} ( t ) d t \approx {\sum }_{0}^{t} \dot{θ} ( t ) {T}_{s} . & ( 3 )\end{matrix}$$

A rotation matrix may then be applied to the determined angle θ of rotation for each electrode 36 to determine the coordinates of each electrode 36, or it may be applied to a set graphical representation of the treatment element. For example, the following rotational matrix may be used:

$$[ \begin{matrix}cos θ & - sin θ \\ sin θ & cos θ\end{matrix} ] [ \begin{matrix}x \\ y\end{matrix} ] = [ \begin{matrix}x cos θ - y sin θ \\ x sin θ + y cos θ\end{matrix} ] . \qquad ( 4 )$$

Similarly, a three-dimensional accelerometer 66 may also provide positional or movement information. Like the gyroscope 64, the accelerometer 66 may be located proximate the treatment array 26, such as within the distal tip 46 of the shaft 28 or on the carrier arm 34, but may also be located, for example, on the catheter handle 18. Although a gyroscope 64 and an accelerometer 66 are shown in FIG. 1A and a gyroscope 64 is shown in FIGS. 2A and 2B, it will be understood that the treatment catheter 12 may include only a gyroscope 64 or both a gyroscope 64 and an accelerometer 66. Unlike a gyroscope, which may provide information about angular velocity, an accelerometer may provide information about linear acceleration (movement of the catheter). Linear acceleration data may be used to enhance the accuracy of the gyroscope data, with additional processing steps. However, an accelerometer may provide information about movement of the electrode array 26 with respect to the target tissue, a reference point, and/or the last lesion created, which may help the user determine the location of the treatment array 26 within the patient's heart. Data from the one or more location tracking elements may be transmitted to the console 16 by a wired or wireless connection.

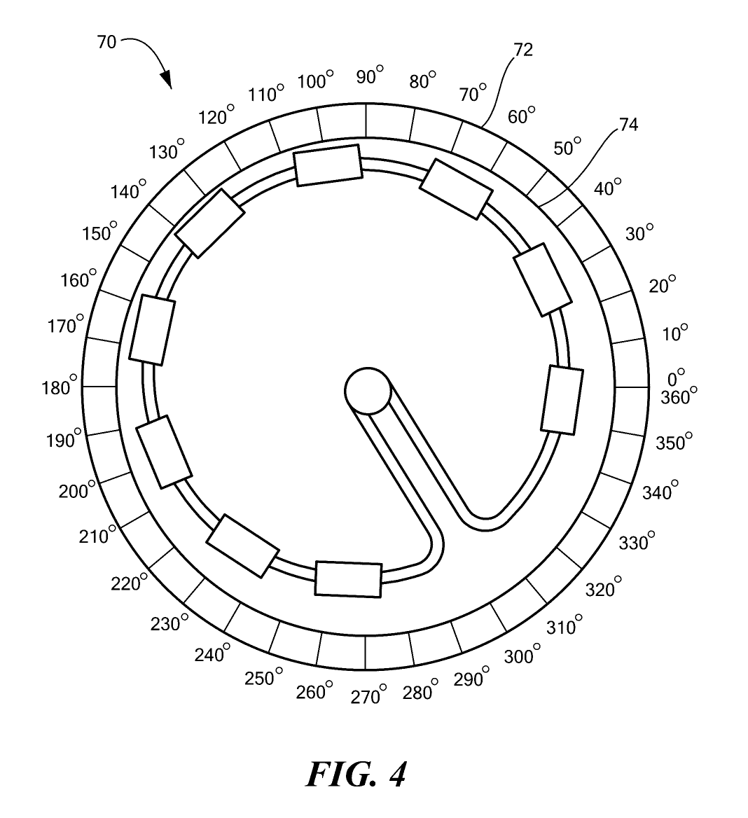

FIG. 4 shows an exemplary treatment array image 70 that may be shown on a system display. The catheter array image 70 may include a fixed reference portion 72. For example, for an electrode array 26 having a circular or semi-circular expanded configuration (such as that shown in FIGS. 1A and 1B), the fixed reference portion 72 may be an outer ring marked with degrees between 0° and 360°. The degrees may be marked incrementally, such as every 10° (as shown in FIG. 4). The treatment array image may also include a stylized representation 74 of a posterior side of the electrode array 26, viewed along the longitudinal axis 32 of the treatment catheter 12 being used. This may allow the user to virtually visualize the electrode array 26 against the target tissue, as if the user were looking down the handle of the catheter 12 toward the target tissue. As the treatment array 26 is rotated by the user in either a clockwise or counter-clockwise direction, the stylized representation 74 may likewise rotate within the fixed reference portion 72. In this way, the user can observe the orientation of the treatment array 26 and the location of each electrode 36. Further, the one or more processors 58 may record the degree corresponding to the location of each electrode and save an orientation history for each electrode 36.

The treatment array image shown in FIG. 4 may be adapted for use with a variety of treatment array configurations. For example, the one or more computers 54 may include software that is able to display any of a plurality of treatment array stylized representations, depending on the user's selection and device being used. As a non-limiting example, the treatment array image may include a stylized representation of a treatment array having electrodes on two crossed carrier arms (for example, similar to the MAAC® catheter (Medtronic, Inc., Minneapolis, Minn.)) or electrodes on three radially arranged carrier arms (for example, similar to the MASC® catheter (Medtronic, Inc., Minneapolis, Minn.)). The system and method disclosed herein may be useful for use with devices having a treatment array that is unsymmetrical or delivers therapy in an unsymmetrical fashion, including those treatment arrays that appear to be symmetrical but for electrode numbering.

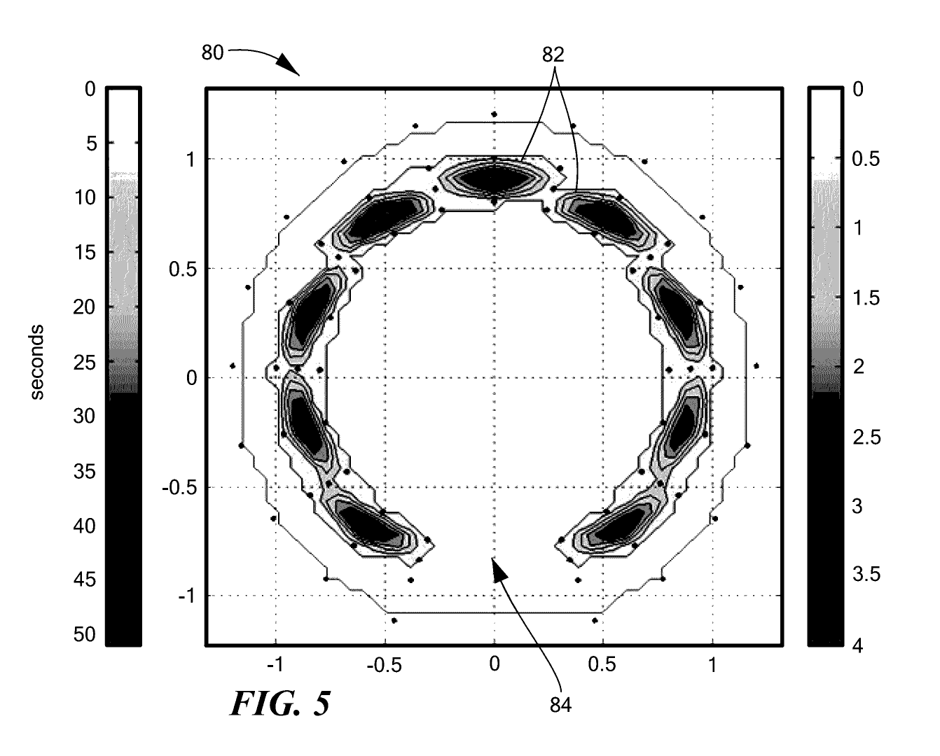

Referring now to FIG. 5, an exemplary graphical representation of a Lesion Prediction Plot (LPP) for an electrode array is shown. Unlike currently known systems that may display electrode data in bar graph form, the LPP 80 may correspond to the size, shape, and configuration of the electrode array 26, and may simulate lesion formation within the target tissue. Further, the LPP 80 may be based at least in part on the effective contact seconds between each electrode and the target tissue. For example, areas of similar amounts of effective contact seconds may be grouped together using isolines, as shown in FIG. 5. Each isoline may be represented by a different color and/or pattern so they are visually distinguishable from each other.

A typical ablation cycle may be 60 seconds, although other treatment lengths may be used, depending on the patient, the area to be ablated, the therapy delivery console, and other variables. As a non-limiting example, the effective contact seconds for each electrode may be determined to be the amount of seconds in the 60-second ablation cycle during which the electrode's temperature is between 50° C. and 65° C. and its power is greater than 3 Watts. Electrode temperatures less than approximately 50° C. and greater than approximately 65° C. and/or power less than 3 Watts may indicate poor electrode-tissue contact, excessive electrode-tissue contact, or electrode malfunction. Conversely, electrode temperature between approximately 50° C. and approximately 65° C. and/or power less than 3 Watts, and for a predetermined threshold of seconds, may indicate the creation of a transmural lesion that is effective in blocking the propagation of aberrant electrical signals through the cardiac tissue. As a non-limiting example, the predetermined threshold of seconds may be 30 effective contact seconds. The temperature and power at each electrode may be determined and monitored by the power generator 52 (for example, a GENius® multi-channel radiofrequency ablation generator (Medtronic, Inc., Minneapolis, Minn.)) and/or computer 54. Using, for example, color-coded isolines 82 corresponding to different amounts of effective contact second, the LPP 80 may provide a simulation of a lesion created by the electrode array 26 within the target tissue, with the areas of the greatest amounts of effective contact seconds corresponding to the deepest or most complete areas of the lesion. For simplicity, each isoline, or groups of isolines, may be correlated to an LPP score (as generally shown in FIG. 5).

The LPP 80 may be updated in real time so the user can visualize the extent of the ablated tissue. Alternatively, the LPP 80 may be generated at the end of the ablation cycle to show a summary of the lesions created by that ablation cycle. If any target ablation sites have not been in contact with one or more electrodes 36 for a sufficient amount of effective contact seconds or energy delivery, the user may reposition the electrode array 26 so that the complete lesion may be created in the target tissue. For example, the electrode array 26 shown in FIGS. 1A and 1B may include nine electrodes 36 (e1-e9), with energy being passed between adjacent electrodes except directly between electrodes e1 and e9. This may leave the corresponding area of target tissue unablated. That is, the lesion may include a gap 84 that corresponds to the space between electrodes e1 and e9. A gap may be defined as an area of target tissue that has an effective contact seconds amount or LPP score that is less than a predetermined threshold. For example, the predetermined threshold may be the lowest desirable score and at which the tissue is still able to conduct aberrant electrical signals. Therefore, if may be desirable to rotate the electrode array 26 so that other electrodes 36 are placed in contact with the lesion gap 84 area. The one or more processors 58 may record the LPP 80 throughout the procedure and may calculate a suggested electrode array 26 rotational orientation as well as, optionally, a location within the patient's heart to create an optimal lesion within the target tissue. The one or more processors 58 may also combine several LPP plots in their respective orientations to create a lesion set of overlaid LPP plots. The lesion set may enable the user to visualize gaps when several ablations are performed around the same location, for example, around one PV ostium or antrum.

In an exemplary method of use, the treatment catheter 12 may be navigated through the patient's vasculature to a target treatment site. For example, the target treatment site may be a pulmonary vein ostium within the left atrium of the heart. The electrode array 26 may be transitioned between a delivery configuration and an expanded configuration (for example, the configuration shown in FIGS. 1A and 1B) and advanced toward the target tissue until the electrode array 26 is in contact with the target tissue. The one or more location tracking elements may then be activated and calibrated. The system may also be calibrated at any point in the procedure before this time, for example, while the catheter is still out of the patient's body or while the catheter is placed in the left atrium at the septal wall. To calibrate a gyroscope 64, for example, the electrode array 26 may be maintained in a steady position with electrode e5 in the “north” position and the gap between electrodes e1 and e9 in the “south” position (for example, as shown in FIG. 2A).

Once the one or more location tracking elements are calibrated, the user may rotate the electrode array 26 to desired orientation. For example, the treatment array image 70 (shown in FIG. 4) may be displayed and updated in real time as the user rotates and moves the electrode array 26. The gyroscope 64 may transmit raw voltage data (proportional to angular velocity) to the one or more processors 58, where the raw data may be used to calculate a degree of rotation. An accelerometer 66 may also transmit linear acceleration data to the processor to be used in calculations to improve the accuracy of the rotational information and also to locate the coordinates of the electrode array (treatment element). Once the electrode array 26 has a desired orientation, energy may be transmitted from the energy generator 52 to the electrode array 26 to ablate the target tissue. Alternatively, if the system is used for a cryotreatment procedure, the circulation of coolant through the treatment element may be initiated. For example, the user may maintain the electrode array 26 in the desired orientation for an ablation cycle of 60 seconds. At the end of the ablation cycle, or during the ablation cycle in real time, the LPP may be displayed as discussed above (FIG. 5).

At the end of each ablation cycle, the LPP may be evaluated, in addition to fluoroscopic images, intracardiac EGM signals, and/or other data. If the LPP and/or other data indicates that the lesion includes any gaps or areas where additional ablation cycles should be performed, the user may rotate and/or reposition the electrode array 26 so that the electrode array 26, or an area of the electrode array 26 other than the area between electrodes e1 and e9, is in contact with the new target tissue area. The one or more processors 58 may receive data from the treatment catheter 12 as described for the first ablation cycle and may compare the effective contact seconds, temperature data, electrode coordinates, and/or other therapy data to that of the first ablation cycle.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An ablation device comprising:
an elongate body having a proximal portion and a distal portion;
a treatment element at the distal portion; and
a gyroscope configured to determine an angular velocity of the treatment element.

2. The ablation device of claim 1, wherein the treatment element includes a carrier arm and a plurality of electrodes, each of the plurality of electrodes being coupled to the carrier arm.

3. The ablation device of claim 2, wherein the carrier arm is configurable into an expanded configuration.

4. The ablation device of claim 2, wherein the treatment element includes at least two carrier arms, the plurality of electrodes being coupled to each of the at least two carrier arms.

5. The ablation device of claim 2, wherein the ablation device further comprising:
a shaft that is slidably disposed within the elongate body, the shaft including a distal portion and a proximal portion.

6. The ablation device of claim 5, wherein the distal portion of the shaft includes a distal tip, the gyroscope being located within the distal tip.

7. The ablation device of claim 1, further including an accelerometer, the gyroscope and the accelerometer being part of a microprocessing unit.

8. The ablation device of claim 7, further including a handle at the proximal portion of the ablation device, wherein the microprocessing unit is located at one of the distal tip of the shaft, a location proximate the at least one electrode, and the handle.

9. A system for evaluating lesion formation in an area of tissue, the system comprising:
an ablation device including:
a treatment assembly having at least one electrode; and
a location tracking element configured to determine an angular velocity and linear acceleration of at least one of the treatment assembly and the ablation device; and
a console including:
a power generator in electrical communication with the treatment assembly; and
at least one processor in communication with the power generator and the location tracking element,
the processor programmed to:
receive ablation data from the power generator;
receive at least one of angular velocity data and linear acceleration data from the location tracking element;
calculate an angle of rotation of the treatment assembly; and
determine a location and movement of the at least one of the treatment assembly and the ablation device.

10. The system of claim 9, wherein the treatment assembly further has a carrier arm and the at least one electrode is a plurality of electrodes, the plurality of electrodes being coupled to the carrier arm.

11. The system of claim 10, wherein the location tracking element is coupled to the carrier arm.

12. The system of claim 11, wherein the ablation device further includes:
an elongate body having a longitudinal axis, a proximal portion, and a distal portion;
a shaft that is slidably movable within and coaxial with the elongate body, the shaft having a proximal portion and a distal portion having a distal tip; and
a handle coupled to the proximal portion of the elongate body.

13. The system of claim 12, wherein the location tracking element is within the distal tip of the shaft.

14. The system of claim 12, wherein the location tracking element is coupled to the handle.

15. The system of claim 9, wherein the location tracking element is one of:
a gyroscope; and
a gyroscope and an accelerometer included in a common microprocessing unit.

16. The system of claim 9, wherein the at least one electrode is a plurality of electrodes and the processor is further programmed to determine a number of effective contact seconds for each of the plurality of electrodes and to generate a graphical representation of the treatment assembly and the number of effective contact seconds for each of the plurality of electrodes.

17. The system of claim 9, wherein the processor is further programmed to receive ablation data from the at least one electrode and to generate a graphical representation of the treatment assembly and ablation data for the at least one electrode.

18. A method for evaluating energy delivery to an area of tissue, the method comprising:
positioning a treatment assembly of a medical device at an area of tissue, the treatment assembly including a plurality of electrodes, the medical device including a location tracking element configured to determine an angular velocity of the treatment assembly;
determining an orientation of the treatment assembly in relation to the area of tissue, the determination being based at least in part on the angular velocity of the treatment assembly;
delivering ablation energy from the plurality of electrodes to the area of tissue for a first ablation cycle;
determining a number of effective contact seconds between each of the plurality of electrodes and the area of tissue from the first ablation cycle; and
generating a lesion prediction display based on the number of effective contact seconds of each of the plurality of electrodes and the area of tissue from the first ablation cycle.

19. The method of claim 18, wherein the lesion prediction display includes a plurality of isolines, each isoline corresponding to a range of effective contact seconds.

20. The method of claim 19, further comprising:
repositioning the treatment assembly such that at least one of the plurality of electrodes is in contact with a portion of the area of tissue for which a number of effective contact seconds from the first ablation cycle is below a threshold number;
delivering ablation energy from the plurality of electrodes to the area of tissue for a second ablation cycle;
determining a number of effective contact seconds between each of the plurality of electrodes and the area of tissue from the second ablation cycle; and
updating the lesion prediction display based on a total number of effective contact seconds of each of the plurality of electrodes and the area of tissue from the first ablation cycle and the second ablation cycle.

21. The method of claim 20, further comprising correlating each of the isolines to a lesion efficacy score.

22. The method of claim 18, wherein each second of ablation time for which one of the plurality of electrodes is operating between 50° C. and 65° C. and at at least 3 Watts is counted as an effective contact second.

23. The method of claim 18, wherein the location tracking element includes a gyroscope.

24. The method of claim 23, wherein the location tracking element further includes an accelerometer, the accelerometer being configured to determine a linear acceleration of the treatment assembly.
